(19) United States
(12) Patent Application Publication
(10) Pub. No.: US 2012/0226321 A1
(43) Pub. Date: Sep. 6, 2012

(54) MODULAR AND NON-MODULAR CORTICAL BUTTRESS DEVICE

(76) Inventor: Eduardo Gonzalez-Hernandez, Coconut Grove, FL (US)

(21) Appl. No.: 13/411,069

(22) Filed: Mar. 2, 2012

Related U.S. Application Data
(60) Provisional application No. 61/448,739, filed on Mar. 3, 2011.

(51) Int. Cl. A61B 17/80

(52) U.S. Cl. 606/286

(57) ABSTRACT

A fixation device serves in facilitating reduction and repair of a fractured bone such as a clavicle or a pelvis. The fixation device includes a bone plate adapted to overlie and contact bone fragments of a fractured bone. The bone plate can include tines extending outwardly from the edge thereof to contact the exterior of and support the bone fragments.

The present application claims the benefit of provisional Application No. 61/448,739, filed Mar. 3, 2011; which is incorporated by reference herein.

### BACKGROUND OF THE INVENTION

1. Field of the Invention

The present invention generally relates to a fixation device used to facilitate reduction and repair of a fractured bone. More particularly, the present invention relates to a bone plate that counteracts deformation forces from surrounding tissue. More specifically, the present invention relates to a bone plate that is configured to maintain the connection of the bone plate with the portions of a fractured bone.

2. Description of the Prior Art

Fractures of bones such as the clavicle can be difficult to repair. Portions of the fractured clavicle are pulled in opposing directions by the interaction thereof with surrounding tissue. For example, deforming forces are applied to the fractured clavicle in at least two directions: (1) force from muscle traction superiorly, and (2) force from muscle traction inferiorly. As such, these deforming forces are transverse to the longitudinal axis of the fractured clavicle, and tend to pull portions of the fractured clavicle apart from one another.

Such deforming forces can destroy the connection between a typical bone plate and the fractured clavicle. Bone screws are used to attach the typical bone plate to the portions of the fractured clavicle, and the bone screws engage the superior cortex bone, the inferior cortex bone, and the medullary canal between the superior and inferior cortex bone. However, the cortex bone is relatively thin, and thus, the threads of the bone screws have a limited amount of bone to engage. That is, the superior and inferior cortex bone provide limited purchase for the threads of the bone screws. Thus, when subjected to the deforming forces, the limited amount of engagement of the bone screws afforded with the cortex bone can lead to failure of the connection between the bone plate and the fractured clavicle. As such, the portions of the fractured clavicle can be pull away from the typical bone plate by the deforming forces.

Therefore, there is a need for a fixation device and method of use associated therewith that is adapted to counteract the above-discussed deformation forces and maintain the connection of a bone plate with the portions of a fractured bone.

### SUMMARY OF THE INVENTION

The present invention in a preferred embodiment contemplates a fixation device for facilitating reduction and repair of a fractured bone, the fixation device including a bone plate adapted to overlie and contact at least two bone fragments of the fractured bone, said bone plate having a body portion includes a first end, a second end, a length between said first and second ends, a first longitudinal edge, a second longitudinal edge, a width between said first and second longitudinal edges, and a bone contacting surface adapted to contact the exteriors of the at least two bone fragments, said body portion including a plurality of bone screw receiving apertures extending therethrough, a first aperture of said plurality of bone screw receiving apertures being adapted to overlie a first bone fragment of the at least two bone fragments and a second aperture of said plurality of bone screw receiving apertures being adapted to overlie a second bone fragment of the at least two bone fragments; at least two bone screws to secure said bone plate to the fractured bone, a first of said at least two bone screws adapted for insertion through said first aperture and into the first of the at least two bone fragments, and a second of said at least two bone screws adapted for insertion through said second aperture and into the second of the at least two bone fragments; and where said bone plate further includes at least two tines extending outwardly from one of said first and second longitudinal edges of said body portion, each of said at least two tines including a bone contacting surface adapted to contact the exterior of and support at least one of the first and second bone fragments, said bone contacting surfaces of said bone plate and said at least two tines being adapted to cradle the fractured bone and resist deformation forces in at least one direction when the fixation device is attached to the fractured bone.

The present invention in a further preferred embodiment contemplates a fixation device for facilitating reduction and repair of a fractured bone, the fixation device including a plate portion adapted to overlie and contact at least two bone fragments of the fractured bone, said plate portion having a first end, a second end, a length between said first and second ends, a first longitudinal edge, a second longitudinal edge, a width between said first and second longitudinal edges, and a bone contacting surface adapted to contact the exteriors of the at least two bone fragments, said plate portion including a plurality of bone screw receiving apertures extending therethrough, said plurality of bone screw receiving apertures being positioned between said first and second longitudinal edges, a first bone screw receiving aperture of said plurality of bone screw receiving apertures being adapted to overlie a first bone fragment of the at least two bone fragments and a second bone screw receiving aperture of said plurality of bone screw receiving apertures being adapted to overlie a second bone fragment of the at least two bone fragments, said plate portion including a first row of at least two apertures positioned along said first longitudinal edge and a second row of at least two apertures positioned along said second longitudinal edge, each of said apertures of said first and second rows of at least two apertures being adapted to receive a buttress pin therein; at least two bone screws to secure said bone plate to the fractured bone, a first of said at least two bone screws being adapted for insertion through said first aperture and into the first of the at least two bone fragments, and a second of said at least two bone screws being adapted for insertion through said second aperture and into the second of the at least two bone fragments; and at least two buttress pins, each of said at least two buttress pins being received in one of said apertures of said first and second rows of at least two apertures, said at least two buttress pins each having a head adapted to engage said plate portion, and a shaft including a bone contacting surface provided to contact the exterior of and support at least one of the first and second bone fragments, said bone contacting surfaces of said plate portion and said at least two buttress pins being adapted to cradle the fractured bone and resist deformation forces in at least one direction, when the fixation device is attached to the fractured bone.

The present invention in yet a further preferred embodiment contemplates a fixation device for facilitating reduction and repair of a fractured bone, the fixation device including a plate portion adapted to overlie and contact at least two bone fragments of the fractured bone, said plate portion having a first end, a second end, a length between said first and second ends, and a bone contacting surface adapted to contact the exteriors of at least two bone fragments, said plate portion including a plurality of bone screw receiving apertures extending therethrough, a first bone screw receiving aperture of said plurality of bone screw receiving apertures being adapted to overlie a first bone fragment of the at least two bone fragments, and a second bone screw receiving aperture of said plurality of bone screw receiving apertures being adapted to overlie a second bone fragment of the at least two bone fragments; at least two bone screws to secure said bone plate to the fractured bone, a first of said at least two bone screws being adapted for insertion through said first aperture and into the first of the at least two bone fragments, and a second of said at least two bone screws being adapted for insertion through said second aperture and into the second of the at least two bone fragments; and at least one clamp adapted to maintain the position of said plate portion with respect to one of the first and second bone fragments, said at least one clamp including a first portion and a second portion spaced apart from one another, where, when said at least one clamp is positioned over said plate portion and the one of the first and second bone fragments, said plate portion and the one of the first and second bone fragments are received between said first and second portions of said clamp.

It is understood that both the foregoing general description and the following detailed description are exemplary and exemplary only, and are not restrictive of the invention as claimed.

### BRIEF DESCRIPTION OF THE DRAWINGS

The accompanying drawings, which are incorporated in and constitute a part of the specification, illustrate preferred embodiments of the invention. Together with the description, they serve to explain the objects, advantages and principles of the invention. In the drawings:

FIG. 1 is a perspective view of a first embodiment of a fixation device according to the present invention positioned with respect to a fractured clavicle;

FIG. 1A is an enlarged perspective view of a fixation device similar to that of FIG. 1 with an additional tine formed thereon;

FIG. 2 is a perspective view of a second embodiment of a fixation device according to the present invention according to the present invention including modular tines;

FIG. 3A is an exploded perspective view of a third embodiment of a fixation device according to the present invention;

FIG. 3B is a perspective view of a fourth embodiment of a fixation device according to the present invention positioned with respect to the fractured clavicle;

FIG. 3C is a cross-sectional view of the fixation devices of FIGS. 3A and 36 with a modified plate portion;

FIG. 3D is a cross-sectional view of the fixation devices of FIGS. 3A and 3B depicting a bone screw received therethrough and into the fractured clavicle;

FIG. 4 is a perspective view of the fixation device of FIG. 3B positioned with respect to a fractured pelvis;

FIG. 5 is a cross-sectional view of the fixation device of FIGS. 3B and 4 in position with respect to the fractured pelvis;

FIG. 6A is an exploded perspective view of a fifth embodiment of a fixation device according to the present invention positioned with respect to a fractured clavicle that includes a c-shaped clamp;

FIG. 6B is a side elevational view of the fixation device of FIG. 6A positioned with respect to the fractured clavicle.

FIG. 7 is a perspective view of the fixation device of FIGS. 6A and 6B including a first modified c-shaped clamp, where the fixation device is positioned with respect to a fractured clavicle; and

FIG. 8 is a perspective view of the fixation device of FIGS. 6A and 6B including a second modified c-shaped clamp, where the fixation device is positioned with respect to a fractured clavicle.

### DETAILED DESCRIPTION OF THE PREFERRED EMBODIMENT

The following description is intended to be representative only and not limiting, and many variations can be anticipated according to these teachings. Reference will now be made in detail to the preferred embodiments of this invention, examples of which are illustrated in the accompanying drawings.

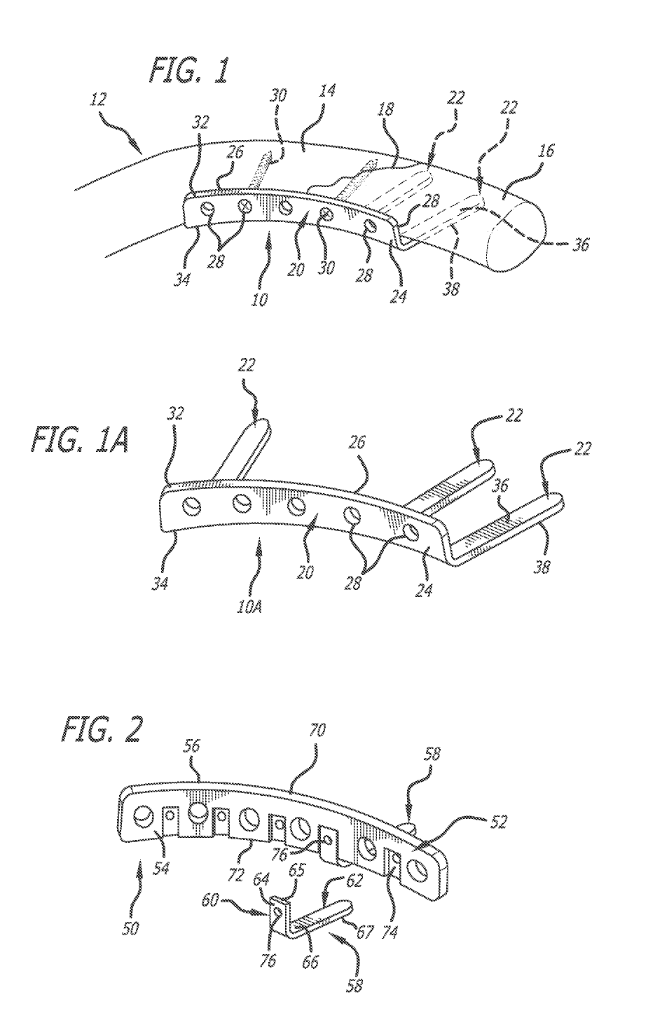

One preferred embodiment of a fixation device of the present invention is generally indicated by the numeral 10 in FIG. 1. Fixation device 10 is formed as a bone plate 11, and is used in stabilizing a fractured clavicle 12. Fractured clavicle 12 includes a first portion 14, a second portion 16, and a fracture line 18 along which first and second portions 14 and 16 are separated. By stabilizing first and second portions 14 and 16 with respect to one another, fixation device 10 serves to facilitate reduction and repair thereof.

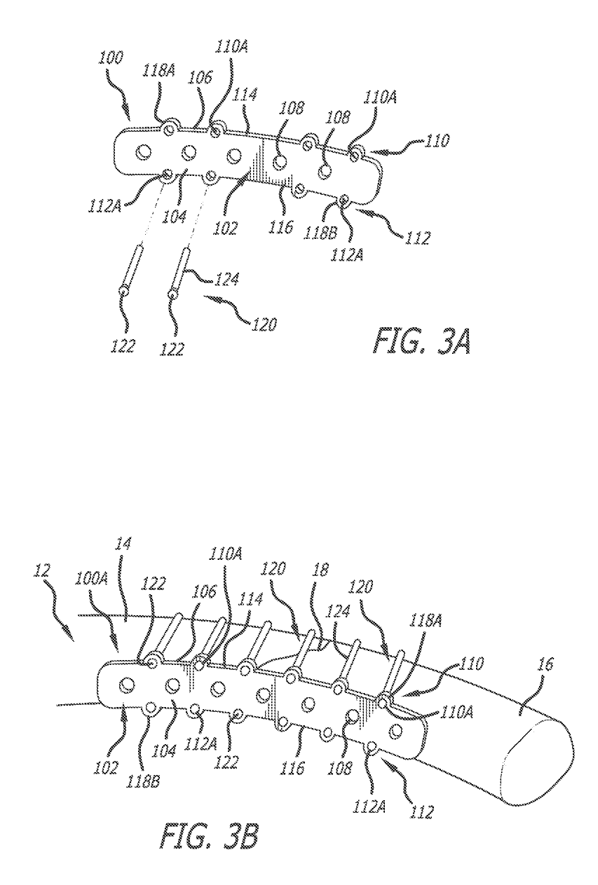

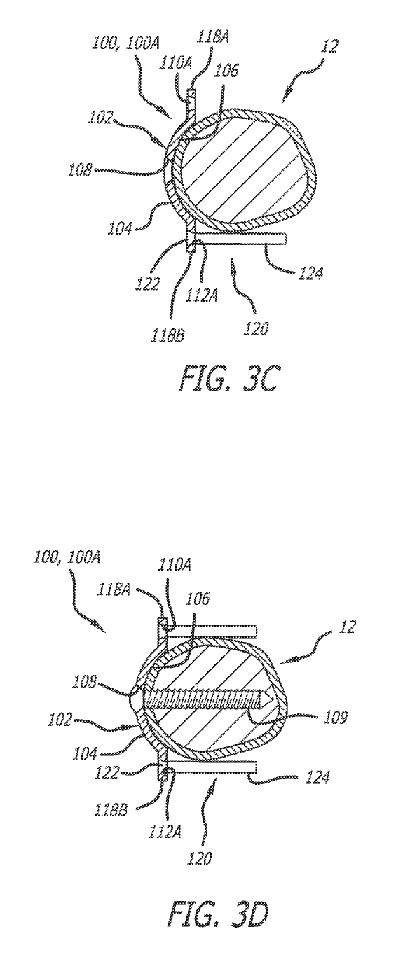

Fixation device 10 includes a body portion 20 for bridging first and second portions 14 and 16, and various tines 22 extending outwardly from body portion 20. Furthermore, body portion 20 includes a first surface 24, a second surface 26, and apertures 28 extending between first and second surfaces 24 and 26. Furthermore, the body portion 20 can be shaped to conform to the shape of the bone requiring reduction and repair (i.e., the fractured clavicle 12 of FIGS. 1, 3B, 3C, and 3D). As depicted in FIG. 1, apertures 28 are spaced along the longitudinal axis of body portion 20 for receiving bone screws 30 therethrough to attach body portion 20 to fractured clavicle 12.

Body portion 20 includes first and second longitudinal edges 32 and 34, and tines 22 can be provided on both of first and second longitudinal edges 32 and 34. As depicted in FIG. 1, tines 22 are provided along second edge 34. Tines 22 each include a first surface 36 and a second surface 38, and extend outwardly from second edge 34. Tines 22 have lengths substantially perpendicular to the longitudinal axis of body portion 20, and have widths substantially aligned with the longitudinal axis of body portion 20. As depicted in FIG. 1, tines 22 provided on second longitudinal edge 34 serve in supporting fractured clavicle 12 inferiorly, and stabilize fractured clavicle 12 by resisting the above-discussed deforming forces.

During use, second surface 26 of body portion 20 along with first surfaces 36 of tines 22 contact fractured clavicle 12. As such, as depicted in FIG. 1, tines 22 are contacted to the inferior cortex of fractured clavicle 12. Furthermore, tines 22 can be curved along the lengths thereof to accommodate the contour of fractured clavicle 12 and facilitate such contact. That is, first surfaces 36 of tines 22 can be concave to facilitate contact with curved surface of fractured clavicle 12.

Tines 22 in combination with body portion 20 cradle fractured clavicle 12, and, in doing so, tines 22 serve to buttress fractured clavicle 12 against the deforming forces in at least one direction. Accordingly, the combination of body portion 20 and tines 22 serves to resist the deforming forces to prevent the connection between fixation device 10 and fractured clavicle 12 from being destroyed.

While fixation device 10 includes two (2) of tines 22 (FIG. 1) spaced along body portion 20, fixation device 10 can be provided with fewer or more tines 22. For example, FIG. 1A depicts a fixation device 10A that includes three (3) of tines 22 spaced therealong. In addition to varying in number, tines 22 also can be provided on first longitudinal edge 32 to support fractured clavicle 12 superiorly. Thus, when tines 22 are provided on both of first and second longitudinal edges 32 and 34, fractured clavicle 12 can be supported superiorly and inferiorly.

FIG. 2 depicts another preferred embodiment of a fixation device of the present invention generally indicated by the numeral 50. Fixation device 50 includes a body (or plate) portion 52 including a first surface 54 and a second surface 56, and body portion 52 serves in bridging first and second portions 14 and 16 of fractured clavicle 12, and incorporates elements similar to fixation devices 10 and 10A. Unlike fixation devices 10 and 10A, however, fixation device 50 includes modular tines 58.

As depicted in FIG. 2, modular fines 58 can be L-shaped, and are removably attached to the body portion 52. Modular tines 68 are initially detached from body portion 20, and can be attached thereto before or after body portion 20 is attached to fractured clavicle 12. Modular fines 58 each include a first portion 60 and a second portion 62 substantially perpendicular to one another. Furthermore, first portion 60 includes surfaces 64 and 65, and second portion 62 includes surfaces 66 and 67.

Modular tines 58 serve the same function as fines 22 associated with fixation devices 10 and 10A, and can be placed similarly along body portion 52. That is, modular tines 58 can be positioned along first and second longitudinal edges 70 and 72 of body portion 52. As such, modular tines 58 can be positioned on body portion 52 to superiorly and inferiorly support fractured clavicle 12 when fixation device 50 is attached thereto.

Body portion 52 includes grooves 74 provided in first surface 54 thereof for receiving first portions 60 of modular tines 58 therein. The depth of grooves 74 and the thickness of first portions 60 afford first surface 54 (of body portion 52) and surface 64 to be flush with one another. Furthermore, fasteners (not shown) can be received through apertures 76 formed in first portions 60 (between surfaces 64 and 65) to secure attachment of modular tine 58 to body portion 52.

Like tines 22, second portions 62 of modular tines 58 have lengths substantially perpendicular to the longitudinal axis of body portion 52, and have widths substantially aligned with the longitudinal axis of body portion 52. During use, second surface 56 of body portion 52 along with surfaces 66 (of second portions 62) of modular tines 58 contact fractured clavicle 12. For example, modular tines 58 can be contacted to the inferior cortex of fractured clavicle 12. Furthermore, second portions 62 of modular tines 58 can be curved along the lengths thereof to accommodate the contour of fractured clavicle 12 and facilitate such contact. That is, surfaces 66 of second portions 62 of modular tines 58 can be concave to facilitate contact with the curved surface of fractured clavicle 12.

Another preferred embodiment of a fixation device of the present invention is generally indicated by the numerals 100 and 100A in FIGS. 3A and 3B, respectively. FIG. 3A depicts an exploded perspective view of fixation device 100. Furthermore, FIG. 3B depicts a perspective view of fixation device 100A positioned with respect to fractured clavicle 12. Fixation devices 100 and 100A are similar to one another, but for the number and placement of apertures provided therein. As such, portions of fixation devices 100 and 100A have identical cross sections therethrough (FIGS. 3C and 3D), and like element numbering will be used in indicating like features if fixation devices 100 and 100A of FIGS. 3A-3D.

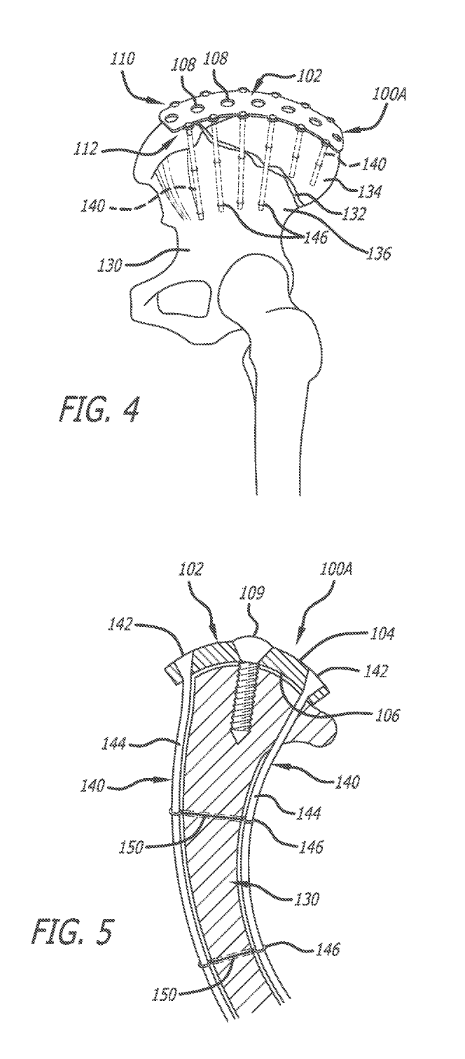

Fixation devices 100 and 100A are formed in part as bone plates, and, like fixation device 10, are used to stabilize fractured clavicle 12. Fixation devices 100 and 100A each include a body (or plate) portion 102 for bridging first and second portions 14 and 16 of fractured clavicle 12 (such as that depicted in FIG. 3B is association with fixation device 100A), and body portion 102 has an upper surface 104 and a lower surface 106. Furthermore, body portion 120 can be shaped to conform to the shape of the bone requiring reduction and repair (i.e., fractured clavicle 12 of FIGS. 3B, 3C, and 3D, or fractured pelvis 130 of FIGS. 4 and 5).

Although not shown in FIGS. 3A and 3B, upper surface 104 and lower surface 106 can be modified to be convex and concave, respectively, as depicted in FIGS. 3C and 3D. The concavity of lower surface 106 can be configured to facilitate contact with the fractured bone such as fractured clavicle 12. Furthermore, the convexity and concavity of upper and lower surfaces 104 and 106 provide body portion 102 with a lower profile when attached to fractured clavicle 12.

Apertures 108 (extending between upper and lower surfaces 104 and 106) are spaced along the longitudinal axis of body portion 102. Apertures 108 of fixation device 100 and 100A are used for receiving bone screws (such as a bone screw 109 depicted in FIG. 3D) therethrough to attach body portion 102 to fractured clavicle 12.

A first row 110 of apertures 110A and a second row 112 of apertures 112A are provided adjacent longitudinal edges 114 and 116, respectively, of body portions 102 of fixation devices 100 and 100A. While apertures 108 are provided to overlie fractured clavicle 12, first and second rows 110 and 112 do not overlie (and hence, are offset from) fractured bone 12 when body portion 102 is positioned with respect thereto.

Furthermore, as depicted in FIGS. 3A and 3B, apertures 110A and 112A are formed through protrusions 118A and 11 SB of body portion 102 formed along longitudinal edges 114 and 116, respectively. However, rather than being formed through protrusions 118A and 118B, apertures 110A and 112A can be formed through other portions of body 102 adjacent longitudinal edges 114 and 116.

Apertures 110A and 112A of first and second rows of apertures 110 and 112 are each configured to receive buttress pins (or rods) 120 therein. As depicted in FIGS. 3B-3D, pins 120 can be substantially perpendicular to the length and width of body portion 102 when received in apertures 110A and 112A. Furthermore, pins 120 can be provided in some or all of apertures 110A and 112A of first and second rows of apertures 110 and 112, and thus, provide fixation devices 100 and 100A with a degree of modularity. Pins 120 of fixation devices 100 and 100A each include a head portion 122 and a shaft portion 124. Shaft portions 124 can be configured to abut and support bone (such as clavicle 12), can be threaded, partially threaded, or non-threaded, and can have various sizes, lengths, and shapes. For example, shaft portions 124 can have cross sections of various shapes. Shaft portions 124 can have cross sections that are at least in part arcuate (e.g., round or ovoid) or that are at least in part rectangular. Furthermore, shaft portions 124 can have lengths approximating the cross-sectional dimensions of fractured clavicle 12. Additionally, shaft portions 124 can be curved along the lengths thereof to facilitate contact with fractured clavicle 12.

Head portions 122 can be threaded to engage threads (not shown) that can be provided in apertures 110A and 112A of first and second rows of apertures 110 and 112. Thus, when body portion 102 is attached to fractured clavicle 12 (using, for example, bone screws 109), pins 120 can be received in apertures 110A and 112A of first and second rows of apertures 110 and 112 to position pins 120 superiorly and inferiorly relative to fractured clavicle 12. If shaft portions 124 are curved along the lengths thereof, threads provided on head portions 122 can be replaced with another mechanism for attaching pins 120 to body portions 100.

As depicted in FIG. 3C, when received in apertures 112A of second row of apertures 112 of fixation devices 100 and 100A, pins 120 function similarly to tines 22 by supporting fractured clavicle 12 inferiorly. That is, pins 120 serve in further stabilizing fractured clavicle 12 by resisting the above-discussed deforming forces in at least one direction. During use, lower surface 106 of body portion 102 along with pins 120 (received in apertures 1124 of second row of apertures 112) contact fractured clavicle 12. As such, pins 120 in combination with body portion 102 of fixation device 100 cradle fractured clavicle 12, and, in doing so, pins 120 serve to buttress fractured clavicle 12 against the deforming forces. Accordingly, like the combination of body portion 20 and tines 22 discussed above in association with fixation device 110, the combination of body portion 102 and pins 120 serves to resist the deforming forces to prevent the connection between fixation device 100 and fractured clavicle 12 from being destroyed.

As depicted in FIG. 3D, when received in apertures 110A and 1124 of first and second rows of apertures 110 and 112 of fixation devices 100 and 100A, pins 120 serve in supporting fractured clavicle 12 superiorly and inferiorly. That is, pins 120 serve in further stabilizing fractured clavicle 12 by resisting the above-discussed deforming forces in at least two directions. During use, lower surface 106 of body portion 102 along with pins 60 (received in apertures 110A and 112A of first and second rows of apertures 110 and 112) contact fractured clavicle 12. As such, pins 120 in combination with body portion 102 of fixation device 100A cradle fractured clavicle 12, and, in doing so, pins 120 serve to buttress fractured clavicle 12 against the deforming forces prevent the connection between fixation device 100A and fractured clavicle 12 from being destroyed.

Besides fractured clavicle 12 depicted in FIG. 3B, fixation devices 100 and 100A can also be used to stabilize fractures of other bones such as the pelvis and shoulder blade. For example, as depicted in FIGS. 4 and 5, fixation device 100A is used in association with a pelvis 130 having a fracture 132 therealong. As depicted in FIG. 4, body portion 102 fixation device 100A is applied over the crest of pelvis 130, and body portion 102 can be attached to a first portion 134 and a second portion 136 of pelvis 130 (using bone screws 109 received through apertures 108, as depicted in FIG. 5) to bridge fracture 132.

Rather than pins 120 depicted in FIG. 3A, extra long pins (or rods) 140 are used in supporting pelvis 130. Like pins 120, pins 140 (FIGS. 4 and 5) each include a head portion 142 and a shaft portion 144, and head portions 142 can be threaded to engage threads (not shown) that can be provided in apertures 110A and 112A of first and second rows 110 and 112. Shaft portions 144 can be deformable to conform to the shape of the adjacent bone. Furthermore, shaft portions 144 can be positioned to be entirely extra-osseous or a segment thereof can penetrate through pelvis 130.

Furthermore, as depicted in FIGS. 4 and 5, tension fasteners 146 can be used to connect pins 140 to one another. For example, as depicted in FIG. 5, holes 150 are drilled through pelvis 130, and tension fasteners 146 are used to connect pins 140 provided on either side of holes 150. Tension fasteners 146 can be wire or sutures wrapped around portions of shaft portions 144. If shaft portions 144 are deformable, tension fasteners 146 can be used to draw shaft portions 144 close to pelvis 130. That is, as tension fasteners 146 are tightened, shaft portions 144 can be drawn closer to pelvis 130. In doing so, pins 140 and tension fasteners 146 form a lattice structure for buttressing pelvis 130.

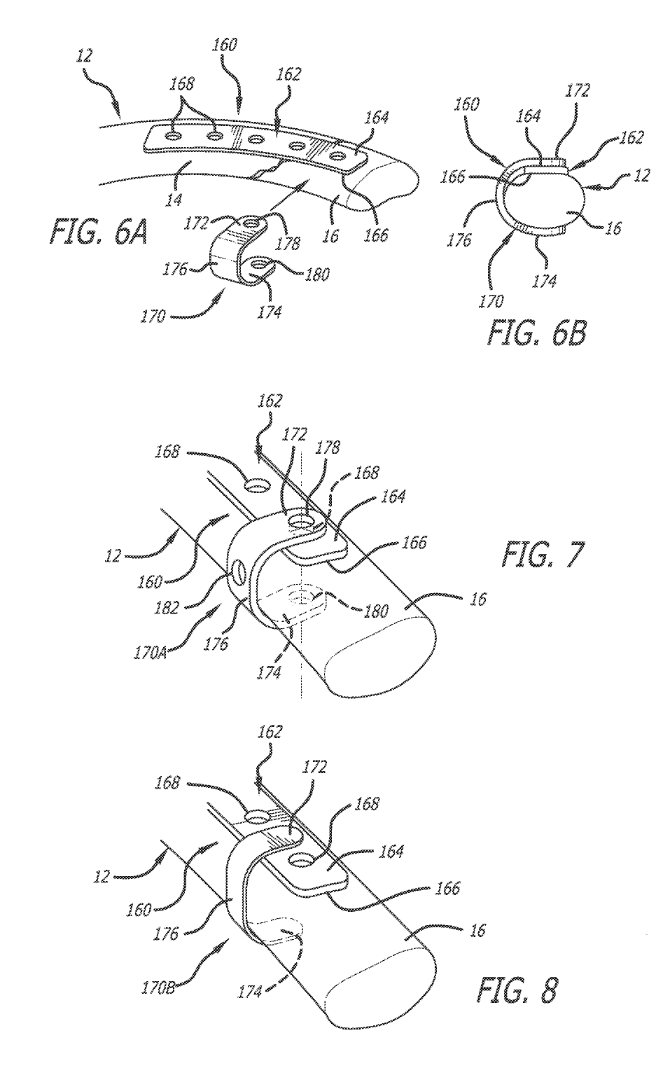

FIGS. 6A and 6B depict another preferred embodiment of a fixation device according to the present invention generally indicated by the numeral 160. As depicted in FIG. 6A, fixation device 160 includes a body (or plate) portion 162 for bridging first and second portions 14 and 16 of fractured clavicle 12, and body portion 162 has an upper surface 164 and a lower surface 166. Apertures 168 (extending between upper and lower surfaces 164 and 166) are spaced along the longitudinal axis of body portion 162. Apertures 168 are used for receiving bone screws (not shown) to facilitate attachment of body portion 162 to fractured clavicle 12.

Fixation device 160 also includes one or more c-shaped clamps 170 to further stabilize fractured clavicle 12. One or more clamps 170 can be positioned relative to body portion 162 and fractured clavicle 12, and provide fixation device 160 with a degree of modularity. That is, clamp 170 can be selectively positioned along body portion 162 and first and second portions 14 and 16 of fractured bone 12. As depicted in FIGS. 6A and 6B, clamp 170 includes a first leg portion 172, a second leg portion 174, and a connecting portion 176 connecting first and second leg portions 172 and 174. Connecting portion 176 has a length permitting first and second leg portions 172 and 174 to fit over fractured clavicle 12 and body portion 162 positioned adjacent fractured clavicle 12. Furthermore, each of first and second leg portions 172 and 174 include opposed surfaces for contacting one of body portion 162 and fractured clavicle 12.

First leg portion 172 can include an aperture 178 formed therein, and second leg portion 174 can include an aperture 180 therein. Furthermore, clamp 170 can be positioned so that aperture 178 is aligned with one of apertures 168 formed in body portion 162. As such, one of the bone screws used to attach body portion 162 to fractured clavicle 12 can be received through aperture 178 of clamp 170 and one of apertures 168 formed in body portion 162. The bone screws can threadably or non-threadably engage body portion 162 and/or clamp 170. When installed, clamp 170 serves in supporting fractured clavicle 12 superiorly and inferiorly. In doing so, clamp 170 serves in buttressing fractured clavicle 12 against the deforming forces to prevent the connection between fixation device 160 and fractured clavicle 12 from being destroyed.

A modified clamp 170A and a modified clamp 170B are depicted in FIGS. 7 and 8, respectively. In comparison to clamp 170, modified clamp 170A (FIG. 7) includes an additional aperture 182 formed through connecting portion 176. A bone screw (not shown) can be received through aperture 182 to engage fractured clavicle 12, and, in doing so, increase the strength of the connection between clamp 170 and fractured clavicle. Furthermore, modified clamp 170B (FIG. 8) includes first and second leg portions 172 and 174, and connecting portion 176 that can be thinner or wider than those of clamp 170. For example, as depicted in FIG. 8, first and second leg portions 172 and 174, and connecting portion 176 of modified clamp 170 are thinner than corresponding portions 172, 174, and 176 in comparison to clamp 170.

Other embodiments of the invention will be apparent to those skilled in the art from consideration of the specification and practice of the invention disclosed herein. Accordingly, it is intended that the specification and examples be considered as exemplary only.

I claim:

1. A fixation device for facilitating reduction and repair of a fractured bone, the fixation device comprising:
a bone plate adapted to overlie and contact at least two bone fragments of the fractured bone, said bone plate having a body portion includes a first end, a second end, a length between said first and second ends, a first longitudinal edge, a second longitudinal edge, a width between said first and second longitudinal edges, and a bone contacting surface adapted to contact the exteriors of the at least two bone fragments, said body portion including a plurality of bone screw receiving apertures extending therethrough, a first aperture of said plurality of bone screw receiving apertures being adapted to overlie a first bone fragment of the at least two bone fragments and a second aperture of said plurality of bone screw receiving apertures being adapted to overlie a second bone fragment of the at least two bone fragments;
at least two bone screws to secure said bone plate to the fractured bone, a first of said at least two bone screws adapted for insertion through said first aperture and into the first of the at least two bone fragments, and a second of said at least two bone screws adapted for insertion through said second aperture and into the second of the at least two bone fragments; and
wherein said bone plate further includes at least two tines extending outwardly from one of said first and second longitudinal edges of said body portion, each of said at least two tines including a bone contacting surface adapted to contact the exterior of and support at least one of the first and second bone fragments, said bone contacting surfaces of said bone plate and said at least two tines being adapted to cradle the fractured bone and resist deformation forces in at least one direction when the fixation device is attached to the fractured bone.

2. The fixation device of claim 1, wherein each of said at least two tines are removably attached to said body portion.

3. The fixation device of claim 1, wherein each of said at least two removably attached tines includes a first portion and a second portion substantially perpendicular to one another, said first portions being adapted to engage said body portion, and said second portion extending outwardly from said one of said first and second longitudinal edges.

4. The fixation device of claim 1, wherein said at least two tines are spaced apart from one another on said one of said first and second longitudinal edges.

5. The fixation device of claim 1, wherein said at least two tines are adapted to resist one of an inferior deformation force and a superior deformation force.

6. The fixation device of claim 1, further comprising an additional two tines extending from the other of said first and second longitudinal edges of said body portion, said at least two tines resisting one of inferior deformation force and superior deformation force, and said additional two tines resisting the other of the inferior deformation force and the superior deformation force.

7. The fixation device of claim 1, wherein one of said at least two tines includes a length extending outwardly from said body portion, the length of said body portion and said length of said one of said at least two tines being substantially perpendicular to one another, said contacting surface of said one of said at least two tines being concave along the length thereof.

8. A fixation device for facilitating reduction and repair of a fractured bone, the fixation device comprising:
a plate portion adapted to overlie and contact at least two bone fragments of the fractured bone, said plate portion having a first end, a second end, a length between said first and second ends, a first longitudinal edge, a second longitudinal edge, a width between said first and second longitudinal edges, and a bone contacting surface adapted to contact the exteriors of the at least two bone fragments, said plate portion including a plurality of bone screw receiving apertures extending therethrough, said plurality of bone screw receiving apertures being positioned between said first and second longitudinal edges, a first bone screw receiving aperture of said plurality of bone screw receiving apertures being adapted to overlie a first bone fragment of the at least two bone fragments and a second bone screw receiving aperture of said plurality of bone screw receiving apertures being adapted to overlie a second bone fragment of the at least two bone fragments, said plate portion including a first row of at least two apertures positioned along said first longitudinal edge and a second row of at least two apertures positioned along said second longitudinal edge, each of said apertures of said first and second rows of at least two apertures being adapted to receive a buttress pin therein;
at least two bone screws to secure said bone plate to the fractured bone, a first of said at least two bone screws being adapted for insertion through said first aperture and into the first of the at least two bone fragments, and a second of said at least two bone screws being adapted for insertion through said second aperture and into the second of the at least two bone fragments; and
at least two buttress pins, each of said at least two buttress pins being received in one of said apertures of said first and second rows of at least two apertures, said at least two buttress pins each having a head adapted to engage said plate portion, and a shaft including a bone contacting surface provided to contact the exterior of and support at least one of the first and second bone fragments, said bone contacting surfaces of said plate portion and said at least two buttress pins being adapted to cradle the fractured bone and resist deformation forces in at least one direction, when the fixation device is attached to the fractured bone.

9. The fixation device of claim 8, wherein said at least two buttress pins are received in said apertures of said first row of at least two apertures, said at least two buttress pins being adapted to resist one of an inferior deformation force and a superior deformation force.

10. The fixation device of claim 8, wherein a first buttress pin of said at least two buttress pins is provided in one of said apertures of said first row of at least two apertures, and a second buttress pin of said at least two buttress pins is provided in one of said apertures of said second row of at least two apertures.

11. The fixation device of claim 10, further comprising at least one tension fastener adapted to extend between said first and second buttress pins, said at least one tension fastener being spaced from said plate portion when the fixation device is attached to the fractured bone.

12. The fixation device of claim 8, wherein said heads of said buttress pins threadably engage the plate portion.

13. The fixation device of claim 11, wherein each of said at least two buttress pins includes a longitudinal axis, the longitudinal axes of said at least two buttress pins being substantially perpendicular to the length and the width of said plate portion when engaged to the plate.

14. The fixation device of claim 8, wherein, when said plate portion is positioned with respect the first and second bone fragments, said first and second rows of at least two apertures are offset from the first and second bone fragments.

15. A fixation device for facilitating reduction and repair of a fractured bone, the fixation device comprising:
a plate portion adapted to overlie and contact at least two bone fragments of the fractured bone, said plate portion having a first end, a second end, a length between said first and second ends, and a bone contacting surface adapted to contact the exteriors of at least two bone fragments, said plate portion including a plurality of bone screw receiving apertures extending therethrough, a first bone screw receiving aperture of said plurality of bone screw receiving apertures being adapted to overlie a first bone fragment of the at least two bone fragments, and a second bone screw receiving aperture of said plurality of bone screw receiving apertures being adapted to overlie a second bone fragment of the at least two bone fragments;
at least two bone screws to secure said bone plate to the fractured bone, a first of said at least two bone screws being adapted for insertion through said first aperture and into the first of the at least two bone fragments, and a second of said at least two bone screws being adapted for insertion through said second aperture and into the second of the at least two bone fragments; and
at least one clamp adapted to maintain the position of said plate portion with respect to one of the first and second bone fragments, said at least one clamp including a first portion and a second portion spaced apart from one another, wherein, when said at least one clamp is positioned over said plate portion and the one of the first and second bone fragments, said plate portion and the one of the first and second bone fragments are received between said first and second portions of said clamp.

16. The fixation device of claim 15, further comprising first and second opposed surfaces formed on said first and second portions, respectively, of said at least one clamp, said opposed surfaces being oriented towards one another, and one of the opposed surfaces being adapted to contact one of said plate portion and the fractured bone, and the other of the opposed surfaces being adapted to contact the other of said plate portion and the fractured bone.

17. The fixation device of claim 15, further comprising a bone screw receiving aperture formed through said first portion of said at least one clamp, said bone screw receiving aperture, when said at least one clamp is positioned over said plate portion, being adapted to communicate with one of said plurality of one screw receiving apertures of said plate portion.

18. The fixation device of claim 15, further comprising a connecting portion joining said first and second portions of said at least one clamp to one another.
